(19) United States
(12) Patent Application Publication
(10) Pub. No.: US 2003/0096782 A1
(43) Pub. Date: May 22, 2003

(54) Expression profiling in the intact human heart

(75) Inventors: Michael R. Bristow, Cherry Hills Village, CO (US); Wayne A. Minobe, Golden, CO (US); Brian D. Lowes, Denver, CO (US); M. Benjamin Perryman, Denver, CO (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701-3271, US

(73) Assignee: The Regents of the University of Colorado

(21) Appl. No.: 10/241,368

(22) Filed: Sep. 11, 2002

Related U.S. Application Data
(60) Provisional application No. 60/318,854, filed on Sep. 11, 2001.

(51) Int. Cl. A61K 48/00; C12Q 1/68

(52) U.S. Cl. 514/44; 435/6

(57) ABSTRACT

Methods for the identification of genes involved in cardiac disease states are provided. The methods compare gene expression between diseased and therapeutically treated patients. Through the identification of new targets, additional methods for drug screening and therapy also are provided.

This application claims benefit of priority to U.S. Provisional Application Serial No. 60/318,854, filed Sep. 11, 2001, the entire contents of which is hereby incorporated by reference without reservation.

The government owns rights in this application pursuant to Grant No. HL48010 from the National Institutes of Health.

### BACKGROUND OF THE INVENTION

A. Field of the Invention

This invention relates to cardiology and molecular biology. In particular, it relates to gene expression profiling, the identification of genes involved in cardiac hypertrophy and associated pathological conditions, and to the treatment of cardiac disease.

B. Description of Related Art

Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to dilated cardiomyopathy, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been elucidated. Understanding these mechanisms is a major concern in the prevention and treatment of cardiac disease and will be crucial as a therapeutic modality in designing new drugs that specifically target cardiac hypertrophy and cardiac heart failure.

Previous approaches have targeted various signaling pathways implicated in cardiac hypertrophy in vitro utilizing cardiomyocytes in culture. However, a major caveat of this approach is that it does not account for physiological relevance in vivo. Other approaches have utilized various drugs that block neurohormonal receptors such as those that mediate the effects of angiotensin II, endothelin-1 and norepinephrine, and assessed the effects on cardiac hypertrophy. The caveat of these approaches is that most often they do not result in acute improvement. A few approaches have targeted the involvement of calcium through examination of calcineurin (Rao et al., 1997; Flanagan et al., 1991; Loh et al., 1996a and Loh et al., 1996b) and sacroendoplasmic reticulum Ca2+ ATPase (Zarain-Herzberg et al., 1999), but the degree to which these pathways are involved in the transduction of various hypertrophic stimuli has not been elucidated. Activation of cell surface receptors for Angiotensin II, and Endothelin-1 leads to subsequent activation of phospholipase C, resulting in the production of diacylglycerol and inositol triphosphate, which in turn results in mobilization of intracellular Ca2+ and activation of protein kinase C (PKC) (Sadoshima et al., 1993; Yamazaki et al., 1996; and Zou et al., 1996). There is also evidence that the Ras and mitogen-activated protein (MAP) kinase pathways are transducers of hypertrophic signals (Force et al., 1996).

Current medical management of cardiac hypertrophy in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn & Bristow, 1996). Other pharmaceutical agents that have been disclosed for treatment of cardiac hypertrophy include angiotensin II receptor antagonists (U.S. Pat. No. 5,604,251) and neuropeptide Y antagonists (International Patent Publication No. WO 98/33791). Despite currently available pharmaceutical compounds, prevention and treatment of cardiac hypertrophy, and subsequent heart failure, continue to present a therapeutic challenge.

Thus, there is a need for the development of new therapeutic strategies in the prevention and treatment of cardiac diseases in humans. In order to develop such strategies, there is a need for intact human models which accurately reflect the physiological and pathological profiles of the disease, thereby allowing identification of novel gene targets for therapeutic intervention. In addition, there is a need for novel assays that allow identification of potential new therapeutic agents for the prevention and treatment of cardiac diseases. Lastly, there is a need to develop a therapeutic strategy that can eliminate interpatient noise generated by experiments that compare different patients with similar phenotypes, allowing a more accurate temporal assessment of genes that are involved in a single patient's disease.

### SUMMARY OF INVENTION

The present invention overcomes the deficiencies in the art by providing a novel approach to identifying genes involved in cardiac disease, and in recovery therefrom. Thus, in accordance with the present invention, there is provided a method of identifying gene involvement in the development, progression and/or maintenance of a disease state comprising (a) obtaining a nucleic acid-containing sample from a first subject suffering from the disease state; (b) obtaining a histologically similar nucleic acid-containing sample from a subject suffering from the disease state, wherein the subject has received a therapy that ameliorates or does not modify the phenotype of the disease state; (c) obtaining gene expression profiles from the samples in steps (a) and (b); and (d) comparing the gene expression profile from the samples in steps (a) and (b); wherein in subjects who exhibit an improvement in phenotype a gene whose expression increases or decreases in the sample of step (b), as compared to the sample of step (a) and to matched samples in subjects who do not exhibit improved phenotype, is identified as being involved in the development, progression and/or maintenance of the disease state. The method may further comprise (e) obtaining a second nucleic acid-containing sample from the subject of step (b)(i) from least at one later time point; (f) obtaining a gene expression profile from the sample in step (e); and (g) comparing the gene expression profile in step (f) to the gene expression profile in step(b)(i).

The disease state may be, but is not limited to, heart failure, cancer, obesity, a neurodegenerative disease, kidney failure, and liver failure. The nucleic acid-containing sample may be a tissue sample. Obtaining expression profiles may comprise PCR, such as RT-PCR. A gene array disposed on a chip may be employed. The method may further comprise comparing the gene expression profile from the samples in steps (a) and/or (b) with the gene expression profile of a subject suffering from the disease state receiving placebo rather than therapy. The method may further comprise comparing the gene expression profile from the samples in steps (a) and/or (b) with the gene expression profile of a histologically similar sample from a healthy individual. The method may further comprise repeating steps (a)-(d) with at least a second subject suffering from the disease state, and comparing the results obtained with the first subject.

In an additional embodiment, there is provided a method of identifying gene involvement in the development, progression and/or maintenance of a disease state of an individual comprising (a) obtaining a nucleic acid-containing sample from a subject suffering from the disease state; (b) obtaining a histologically similar nucleic acid-containing sample from the subject of step (a) from at least one later time point, prior to which the subject has received a therapy that ameliorates the phenotype of the disease state; (c) obtaining gene expression profiles from the samples in steps (a) and (b); and (d) comparing the gene expression profile from the samples in steps (a) and (b), wherein a gene whose expression increases or decreases in the sample of step (b), as compared to the samples of step (a), is identified as being involved in the development, progression and/or maintenance of the disease state. The method may further comprise repeating step (b) at a second later time point. The method also may further comprise (e) obtaining a histologically similar nucleic acid-containing sample from a subject suffering from the disease state, wherein the subject has received a therapy that does not ameliorate the phenotype of the disease state; (f) obtaining a gene expression profile from the sample in step (e); and (g) comparing the gene expression profile in step (f) to the gene expression profile in step(b).

In another embodiment, there is provided a method for assessing the efficacy of a cardiac disease therapy comprising (a) obtaining a first cardiac tissue sample from a first subject suffering from a cardiac disease; (b) treating the first subject with a candidate therapy; (c) obtaining a second cardiac tissue sample from the first subject following treatment; and (d) comparing the expression of one or more indicator genes in the first and second samples, the one or more indicator genes as listed in Table 1, wherein a change in the expression of one or more indicator genes indicates that the candidate therapy is effective at treating cardiac disease in the first subject. The methods may rely on the use of one, two, three, four, five, six, seven, eight, nine, ten, 15, 20, 25, 30, 35, 40, 45, 50, 75, 100, 125 or all of the genes of Table 1. The methods may also rely on at least one down-regulated and at least one up-regulated gene, at least two of each class, at least three of each class, at least four of each class or at least five of each class. Specific genes of interest include α-MyHC, MEK5, extracellular matrix (ECM) producing or regulating genes, Shaker-type, delayed rectifier (Kv1.1) voltage-sensitive potassium channel beta subunit (β1, or KCNA1B), and collagenase IV (also known as MMP2).

The method may further comprise comparing the gene expression profile from the samples in steps (a) and/or (b) with the gene expression profile of a cardiac tissue sample from a healthy individual. The method may further comprise comparing the gene expression profile from the samples in steps (a) and/or (b) with the gene expression profile of a cardiac tissue sample from a second subject suffering from cardiac disease receiving a placebo rather than therapy. The method may further comprise repeating steps (a)-(d) with at least a second subject suffering from cardiac disease, and comparing the results obtained with the first subject. The method may further comprise repeating steps (a)-(d) on the first subject after altering the dose or dosing regimen of the candidate therapy.

In yet another embodiment, there is provided a method of screening a candidate substance for their ability to modulate the activity of one or more cardiac disease gene products in cardiac cells comprising (a) providing a myocyte; (b) contacting the myocyte with the candidate substance; and (c) measuring the activity of one or more gene products selected from the group consisting of Table 1, wherein a change in the activity of one or more gene products selected from the group consisting of Table 1, as compared to the activity in a myocyte not contacted with the candidate substance, indicates that the candidate substance is a modulator of the activity of one or more cardiac disease gene products. The methods may rely on the use of one, two, three, four, five, six, seven, eight, nine, ten, 15, 20, 25, 30, 35, 40, 45, 50, 75, 100, 125 or all of the genes of Table 1. The methods may also rely on at least one down-regulated and at least one up-regulated gene, at least two of each class, at least three of each class, at least four of each class or at least five of each class. Specific genes of interest include α-MyHC, MEK5, extracellular matrix (ECM) producing or regulating genes, Shaker-type, delayed rectifier (Kv1.1) voltage-sensitive potassium channel beta subunit (β1, or KCNA1B), and collagenase IV (also known as MMP2).

Measuring the activity may comprise measuring mRNA levels, optionally comprising RT-PCR, measuring protein levels, or measuring enzyme activity. The myocyte may be a cardiomyocyte. The myocyte may be contacted in culture or in a non-human animal. The myocyte may be transformed with an expression construct comprising a screenable marker gene under the control of a promoter derived from a gene selected from Table 1. The myocyte may exhibit cardiac disease-like gene expression patterns.

In still yet another embodiment, there is provided a method for treating cardiac disease comprising administering to a subject in need thereof a substance that inhibits the activity of one or more of the down-regulated gene products listed in Table 1. The methods may rely on the use of one, two, three, four, five, six, seven, eight, nine, ten, 15, 20, 25, 30, 35, 40, 45, 50, 75, or 100 of the downregulated genes of Table 1. The substance may be a protein or a nucleic acid expression construct. The nucleic acid expression construct may encode an antisense construct or ribozyme. The substance may be a small molecule or organo-pharmaceutical.

In a further embodiment, there is provided a method for treating cardiac disease comprising administering to a subject in need thereof a substance that increases the activity of one or more of the upregulated gene products in Table 1. The methods may rely on the use of one, two, three, four, five, six, seven, eight, nine, ten, or 11 of the upregulated genes of Table 1. The substance may be a protein or a nucleic acid expression construct. The nucleic acid expression construct may encode one or more of the upregulated gene products in Table 1. The substance may be a small molecule or organo-pharmaceutical.

### DESCRIPTION OF ILLUSTRATIVE EMBODIMENTS

### A. The Present Invention

This present invention provides improved methods for gene expression profiling to identify genes involved in diseases such as cardiac heart failure and associated pathological conditions. In one embodiment, genes involved in cardiac heart failure may be identified by using serial studies upon diseased and treated tissues. However, the invention also applies to other diseases which include cancer, obesity, a neurodegenerative disease, kidney failure, liver failure, and others, as will be appreciated by the skilled artisan.

Overall, the major advantages, as compared to current approaches, that the invention herein addresses are: (1) that gene expression is combinatorial and therefore requires the intact heart organ versus in vitro culture systems; (2) that the current explanted heart model has thus far proven to be problematic due to endstage disease, organ donors as controls, inability to perform interventions, and marked variability between subjects within apparent phenotypic groups; and (3) that the invention greatly decreases intersubject variation in data analysis by allowing serial analysis of a single subject taken at multiple time points during disease evolution or regression. Further, using the intact heart serves to provide a more physiological and pathological relevant organ model system. Another important aspect of the invention is to compare diseased tissue with successfully treated tissue, as opposed to healthy tissue. When used in conjunction with powerful techniques such as gene chip array and RT-PCR, the methods described herein provide much more robust data than typical approaches.

The invention further provides a method for screening cardiomyocytes and intact organisms with various candidate substances to further assess the activity of the substances on newly identified cardiac disease targets. The invention also provides a method for treating target cardiac diseases and associated pathological conditions.

In the attached Table 1, a number of gene targets are provided which, in accordance with the present invention, have been identified as being differentially regulated in conjunction with diseased cardiac versus successfully treated cardiac tissue.

### B. Assaying for Relative Gene Expression

The present invention, in various embodiments, involves assaying for gene expression. There are a wide variety of methods for assessing gene expression, most of which are reliant on hybrdization analysis. In specific embodiments, template-based amplification methods are used to generate (quantitatively) detectable amounts of gene products, which are assessed in various manners. The following techniques and reagents will be useful in accordance with the present invention:

1. Obtaining Samples of Intact Cardiac Tissue

Endomyocardial biopsy is an accepted, useful invasive tool for the analysis of the endomyocardium at both the cellular and subcellular levels. Endomyocardial samples may be obtained by several techniques that are well established. The conventional methods involve retrieving biopsy samples from the left internal jugular or femoral vein or by use of the right or left internal jugular or subclavian. Left or right ventricular endomyocardial biopsy is dependent upon which side of the heart is predominantly involved in cardiac disease at the time of diagnosis, or when one biopsy is more successful on a particular side.

Another approach may involve a more conventional procedure utilizing a 7 Fr 35 cm sheath and dilator system placed into the right ventricle over a balloon-tipped catheter. After the sheath is positioned via either internal jugular or subclavian vein, multiple samples may be obtained using standard bioptomes. Another approach involves a two-dimensional echocardiography wherein a transducer is placed at the apex and in the subcostal area and four-chamber views were utilized. The bioptome then enters the right atrium and crosses the tricuspid valve to the right ventricle. The catheter is then manipulated under two-dimensional echocardiography and the tip's position strictly adapted, using two different classic views before sampling.

2. Amplification Methodology

A useful technique in working with nucleic acids involves amplification. Amplifications are usually template-dependent, meaning that they rely on the existence of a template strand to make additional copies of the template. Primers, short nucleic acids that are capable of priming the synthesis of a nascent nucleic acid in a template-dependent process, are hybridized to the template strand. Typically, primers are from ten to thirty base pairs in length, but longer sequences can be employed. Primers may be provided in double-stranded and/or single-stranded form, although the single-stranded form generally is preferred.

Often, pairs of primers are designed to selectively hybridize to distinct regions of a template nucleic acid, and are contacted under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

PCR: A number of template dependent processes are available to amplify the oligonucleotide sequences present in a given template sample. One of the best known amplification methods is the polymerase chain reaction (referred to as PCRTM) which is described in detail in U.S. Pat. Nos. 4,683,195, 4,683,202 and 4,800,159, and in Innis et al., 1988, each of which is incorporated herein by reference in their entirety. In PCR™, pairs of primers that selectively hybridize to nucleic acids are used under conditions that permit selective hybridization. The term primer, as used herein, encompasses any nucleic acid that is capable of priming the synthesis of a nascent nucleic acid in a template-dependent process. Primers may be provided in double-stranded or single-stranded form, although the single-stranded form is preferred.

The primers are used in any one of a number of template dependent processes to amplify the target-gene sequences present in a given template sample. One of the best known amplification methods is PCR™ which is described in detail in U.S. Pat. Nos. 4,683,195, 4,683,202 and 4,800,159, each incorporated herein by reference.

In PCR™, two primer sequences are prepared which are complementary to regions on opposite complementary strands of the target-gene(s) sequence. The primers will hybridize to form a nucleic-acid:primer complex if the target-gene(s) sequence is present in a sample. An excess of deoxyribonucleoside triphosphates are added to a reaction mixture along with a DNA polymerase, e.g., Taq polymerase, that facilitates template-dependent nucleic acid synthesis.

If the target-gene(s) sequence:primer complex has been formed, the polymerase will cause the primers to be extended along the target-gene(s) sequence by adding on nucleotides. By raising and lowering the temperature of the reaction mixture, the extended primers will dissociate from the target-gene(s) to form reaction products, excess primers will bind to the target-gene(s) and to the reaction products and the process is repeated. These multiple rounds of amplification, referred to as “cycles,” are conducted until a sufficient amount of amplification product is produced.

A reverse transcriptase PCR™amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641, filed Dec. 21, 1990.

LCR: Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

Qbeta Replicase: Qbeta Replicase, described in PCT Patent Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which can then be detected.

Isothermal Amplification: An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Such an amplification method is described by Walker et al. (1992).

Strand Displacement Amplification: Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

Cyclic Probe Reaction: Target specific sequences can also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Transcription-Based Amplification: Other nucleic acid amplification procedures include transcription-based amplification systems (TAS), including nucleic acid sequence based amplification (NASBA) and 3SR, Kwoh et al. (1989); PCT Application WO 88/10315, 1989).

In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Other Amplification Methods: Other amplification methods, as described in British Patent Application No. GB 2,202,328, and in PCT Application No. PCT/US89/01025, each incorporated herein by reference, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Davey et al., European Patent Application No. 329 822 (incorporated herein by reference) disclose a nucleic acid amplification process involving cyclically synthesizing single-stranded RNA (“ssRNA”), ssDNA, and double-stranded DNA (dsDNA), which may be used in accordance with the present invention.

The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “race” and “one-sided PCR™” (Frohman, 1990; Ohara et al., 1989, each herein incorporated by reference). Methods based on ligation of two (or more) oligonucleotides in the presence of nucleic acid having the sequence of the resulting “di-oligonucleotide”, thereby amplifying the di-oligonucleotide, also may be used in the amplification step of the present invention, Wu et al. (1989).

2. Chips and Arrays

DNA arrays and gene chip technology provides a means of rapidly screening a large number of DNA samples for their ability to hybridize to a variety of single stranded DNA probes immobilized on a solid substrate. Specifically contemplated are chip-based DNA technologies such as those described by Hacia et al. (1996) and Shoemaker et al. (1996). These techniques involve quantitative methods for analyzing large numbers of genes rapidly and accurately. The technology capitalizes on the complementary binding properties of single stranded DNA to screen DNA samples by hybridization. Pease et al. (1994); Fodor et al. (1991). Basically, a DNA array or gene chip consists of a solid substrate upon which an array of single stranded DNA molecules have been attached. For screening, the chip or array is contacted with a single stranded DNA sample which is allowed to hybridize under stringent conditions. The chip or array is then scanned to determine which probes have hybridized. In a particular embodiment of the instant invention, a gene chip or DNA array would comprise probes specific for chromosomal changes evidencing the development of a neoplastic or preneoplastic phenotype. In the context of this embodiment, such probes could include synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832 which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal, for example, luminescence. The target nucleic acid may be immobilized onto the integrated microchip that also supports a phototransducer and related detection circuitry. Alternatively, a gene probe may be immobilized onto a membrane or filter which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′-end of the molecule during DNA synthesis. DNA may be bound directly to membranes using ultraviolet radiation. With nitrocellous membranes, the DNA probes are spotted onto the membranes. A UV light source (Stratalinker,™ Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

3. Separation Techniques

It may be desirable to separate nucleic acid products from other materials, such as template and excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

### C. Screening For Modulators of the Protein Function

The present invention further comprises methods for identifying modulators of the function of the gene targets identified in Table 1. These assays may comprise random screening of large libraries of candidate substances; alternatively, the assays may be used to focus on particular classes of compounds selected with an eye towards structural attributes that are believed to make them more likely to modulate the function or expression of target genes.

To identify a modulator, one generally will determine the expression or activity of a target gene in the presence and absence of the candidate substance, a modulator defined as any substance that alters function or expression. Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein, the term “candidate substance” refers to any molecule that may potentially inhibit or enhance activity or expression of a target gene. The candidate substance may be a protein or fragment thereof, a small molecule, a nucleic acid molecule or expression construct. It may be that the most useful pharmacological compounds will be compounds that are structurally related to a target gene or a binding partner or substrate therefore. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be an ideal candidate inhibitor.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

2. In Cyto Assays

The present invention contemplates the screening of compounds for their ability to modulate target genes in cells. Various cell lines can be utilized for such screening assays, including cells specifically engineered for this purpose. Engineering may include putting screenable marker genes under the control of a promoter derived from a target gene.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

3. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species. In particular, the assay contemplates the use of humans in clinical trials.

In such assays, one or more candidate substances are administered to a subject, and the ability of the candidate substance(s) to alter one or more target gene activities, as compared to a similar animal not treated with the candidate substance(s), identifies a modulator. Test subjects may have natural or artificially induced disease states, e.g., cardiac hypertrophy, heart failure, etc.

Treatment of these subjects with test compounds will involve the administration of the compound, in an appropriate form, to the subject. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

### D. Therapeutic Agents

1. Targeting Cardiac Disease Genes

a. Protein Expressing Sequences

In one embodiment, one may modulate the expression of selected target genes by providing a therapeutic transgene that expresses a therapeutic polynucleotide. In a first embodiment, a gene encoding a target gene product for which increased expression is desired may be used. Alternatively, a gene encoding a single chain antibody that binds to a target gene for which reduced activity is desired may be used. In order to express such molecules, one must associate the selected nucleic acid in conjunction with proper regulatory machinery, and then one must deliver the construct to a target cell. These aspects of the invention are addressed below.

i. Vectors for Cloning, Gene Transfer and Expression

Within certain embodiments expression vectors are employed to express a therapeutic gene product. Expression requires that appropriate signals be provided in the vectors, which include various regulatory elements, such as enhancers/promoters from both viral and mammalian sources that drive expression of the genes of interest in host cells. Elements designed to optimize messenger RNA stability and translatability in host cells also are defined.

Regulatory Elements. Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used herein to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this type of module is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

Of particular interest are muscle specific promoters, and more particularly, cardiac specific promoters. These include the myosin light chain-2 promoter (Franz et al., 1994; Kelly et al., 1995), the alpha actin promoter (Moss et al., 1996), the troponin 1 promoter (Bhavsar et al., 1996); the Na+/Ca2+ exchanger promoter (Barnes et al., 1997), the dystrophin promoter (Kimura et al., 1997), the creatine kinase promoter (Ritchie, M. E., 1996), the alpha7 integrin promoter (Ziober & Kramer, 1996), the brain natriuretic peptide promoter (LaPointe et al., 1996), the αB-crystallin/small heat shock protein promoter (Gopal-Srivastava, R., 1995), and alpha myosin heavy chain promoter (Yamauchi-Takihara et al., 1989) and the ANF promoter (LaPointe et al., 1988).

Enhancers are genetic elements that increase transcription from a promoter located at a distant position on the same molecule of DNA. Enhancers are organized much like promoters, that is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

Selectable Markers. In certain embodiments of the invention, the cells contain nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

Multigene Constructs and IRES. In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well as IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins encoded by independent genes, intracellular or membrane-bound proteins and selectable markers. In this way, expression of several proteins can be simultaneously engineered into a cell with a single construct and a single selectable marker.

ii. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 109-1012 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varnus et al., 1981). Another concern with the use of defective retrovirus vectors is the potential appearance of wild-type replication-competent virus in the packaging cells. This can result from recombination events in which the intact- sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

b. Non-Protein-Expressing Sequences

In certain embodiments, the one may desire to inhibit the expression of a given target gene. This may be accomplished using transgenes that are not expressed as protein, i.e., transcribed but not translated. DNA may be introduced into organisms for the purpose of expressing RNA transcripts that function to affect phenotype yet are not translated into protein. Two examples are antisense RNA and RNA with ribozyme activity. Both may serve possible functions in reducing or eliminating expression of native or introduced genes. However, as detailed below, DNA need not be expressed to effect the phenotype of an organism.

i. Antisense RNA

In certain aspects, a therapeutic transgene may express an antisense message. Nucleic acids, may be constructed or isolated which, when transcribed, produce antisense RNA that is complementary to all or part(s) of a targeted messenger RNA(s). The antisense RNA reduces production of the polypeptide product of the messenger RNA. The polypeptide product may be any protein encoded by the cell's genome. The aforementioned genes will be referred to as antisense genes. An antisense gene may thus be introduced into a cell by transformation methods to produce a novel transgenic cell or organism with reduced expression of a selected protein of interest. For example, the protein may be an enzyme that catalyzes a reaction in the cell or organism. Reduction of the enzyme activity may reduce or eliminate products of the reaction which include any enzymatically synthesized compound in the cell or organism such as fatty acids, amino acids, carbohydrates, nucleic acids and the like.

Alternatively, in a non-limiting example such as the transformation of a plant cell, the protein may be a storage protein, such as a zein, or a structural protein, the decreased expression of which may lead to changes in seed amino acid composition or plant morphological changes respectively. The possibilities cited above are provided only by way of example and do not represent the full range of applications.

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example of an antisense construct is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit expression, as described in U.S. Pat. No. 5,891,625. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

ii. Ribozymes

In other aspects, the therapeutic transgene may produce a ribozyme. Nucleic acids may be constructed or isolated which, when transcribed, produce RNA enzymes (ribozymes) that can act as endoribonucleases and catalyze the cleavage of RNA molecules with selected sequences. The cleavage of selected messenger RNAs can result in the reduced production of their encoded polypeptide products. These genes may be used to prepare one or more cells, tissues or organisms that carry them. The transgenic cells, tissues or organisms may possess reduced levels of polypeptides including, but not limited to, the polypeptides cited above.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes.

Several different ribozyme motifs have been described with RNA cleavage activity (Symons, 1992). Examples include sequences from the Group I self splicing introns including Tobacco Ringspot Virus (Prody et al., 1986), Avocado Sunblotch Viroid (Palukaitis et al., 1979), and Lucerne Transient Streak Virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992, Yuan and Altman, 1994, U.S. Pat. Nos. 5,168,053 and 5,624,824), hairpin ribozyme structures (Berzal-Herranz et al., 1992; Chowrira et al., 1993) and Hepatitis Delta virus based ribozymes (U.S. Pat. No. 5,625,047). The general design and optimization of ribozyme directed RNA cleavage activity has been discussed in detail (Haseloff and Gerlach, 1988, Symons, 1992, Chowrira et al., 1994; Thompson et al., 1995).

The other variable in ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA where a uracil (U) is followed by either an adenine, cytosine or uracil (A, C or U) (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in down regulating a given gene is simply a matter of preparing and testing a given sequence, and is a routinely practiced “screening” method known to those of skill in the art.

2. Combination Therapies

In order to increase the effectiveness of a given therapy, it may be desirable to combine these compositions and methods of the invention with an agent effective in the treatment of vascular or cardiovascular disease or disorder. In some embodiments, it is contemplated that a conventional therapy or agent, including but not limited to, a pharmacological therapeutic agent, a surgical therapeutic agent (e.g., a surgical procedure) or a combination thereof, may be combined with treatment directed to a gene target. In a non-limiting example, a therapeutic benefit comprises reduced hypertension in a vascular tissue, or reduced restenosis following vascular or cardiovascular intervention, such as occurs during a medical or surgical procedure. Thus, in certain embodiments, a therapeutic method of the present invention may comprise modulating the expression of a gene in Table 1 in combination with another therapeutic agent.

This process may involve contacting the cell(s) with an agent(s) and the target gene modulation at the same time or within a period of time wherein separate administration of the modulator and an agent to a cell, tissue or organism produces a desired therapeutic benefit. The terms “contacted” and “exposed,” when applied to a cell, tissue or organism, are used herein to describe the process by which a modulator and/or therapeutic agent is delivered to a target cell, tissue or organism or is placed in direct juxtaposition with the target cell, tissue or organism. The cell, tissue or organism may be contacted (e.g., by adminstration) with a single composition or pharmacological formulation that includes both a modulator and one or more agents, or by contacting the cell with two or more distinct compositions or formulations, wherein one composition includes a modulator and the other includes one or more agents.

The gene modulator may precede, be co-current with and/or follow the other agent(s) by intervals ranging from minutes to weeks. In embodiments where the modulator and other agent(s) are applied separately to a cell, tissue or organism, one would generally ensure that a significant period of time did not expire between the time of each delivery, such that the modulator and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the modulator. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 12 hours, about 18 hours, about 24 hours, about 36 hours, about 48 hours, about 1 day, about 2 days, about 3 days, about 4 days, about 5 days, about 6 days, about 7 days, about 14 days, about 21 days, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, or about 12 months, and any range derivable therein, prior to and/or after administering the modulator.

Various combination regimens of the modulator and one or more agents may be employed. Non-limiting examples of such combinations are shown below, wherein a composition modulator is “A” and the other agent is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of modulators to a cell, tissue or organism may follow general protocols for the administration of vascular or cardiovascular therapeutics, taking into account the toxicity, if any. It is expected that the treatment cycles would be repeated as necessary. In particular embodiments, it is contemplated that various additional agents may be applied in any combination with the present invention.

3. Pharmacological Therapeutic Agents

Pharmacological therapeutic agents and methods of administration, dosages, etc., are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In addition, it should be noted that any of the following may be used to develop new sets of cardiac therapy target genes as β-blockers were used in the present examples (see below). While it is expected that many of these genes may overlap, new gene targets likely can be developed.

a. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

i. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

ii. Resins/Bile Acid Sequesterants

Non-limiting examples of resins/bile acid sequesterants include cholestyramine (cholybar, questran), colestipol (colestid) and polidexide.

iii. HMG CoA Reductase Inhibitors

Non-limiting examples of HMG CoA reductase inhibitors include lovastatin (mevacor), pravastatin (pravochol) or simvastatin (zocor).

iv. Nicotinic Acid Derivatives

Non-limiting examples of nicotinic acid derivatives include nicotinate, acepimox, niceritrol, nicoclonate, nicomol and oxiniacic acid.

v. Thryroid Hormones and Analogs

Non-limiting examples of thyroid hormones and analogs thereof include etoroxate, thyropropic acid and thyroxine.

vi. Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

b. Antiarteriosclerotics

Non-limiting examples of an antiarteriosclerotic include pyridinol carbamate.

c. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

i. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

ii. Antiplatelet Agents

Non-limiting examples of antiplatelet agents include aspirin, a dextran, dipyridamole (persantin), heparin, sulfinpyranone (anturane) and ticlopidine (ticlid).

iii. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

d. Blood Coagulants

In certain embodiments wherein a patient is suffering from a hemmorage or an increased likelyhood of hemmoraging, an agent that may enhance blood coagulation may be used. Non-limiting examples of a blood coagulation promoting agent include thrombolytic agent antagonists and anticoagulant antagonists.

i. Anticoagulant Antagonists

Non-limiting examples of anticoagulant antagonists include protamine and vitamine K1.

ii. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

e. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

i. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).

ii. Beta Blockers

Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

iii. Repolarization Prolonging Agents

Non-limiting examples of an agent that prolong repolarization, also known as a Class III antiarrhythmic agent, include amiodarone (cordarone) and sotalol (betapace).

iv. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.

v. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

f. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

i. Alpha Blockers

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

ii. Alpha/Beta Blockers

In certain embodiments, an antihypertensive agent is both an alpha and beta adrenergic antagonist. Non-limiting examples of an alpha/beta blocker comprise labetalol (normodyne, trandate).

iii. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

iv. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

v. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimefylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

vi. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

g. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

h. Treatment Agents for Congestive Heart Failure

Non-limiting examples of agents for the treatment of congestive heart failure include anti-angiotension II agents, afterload-preload reduction treatment, diuretics and inotropic agents.

i. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

ii. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene)or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.

iii. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

i. Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

4. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Dynamic expression profiling was performed in the intact human heart in 8 subjects with idiopathic dilated cardiomyopathy (IDC), on RNA extracted from RV septal endomyocardial biopsies performed at baseline and after 6 months treatment with β-blocking agents (n=6) or placebo (n=2). Affmetrix™ U95a GeneChips, which contain 12,625 gene sequences/chip, were used for expression profiling. All 6 subjects treated with β-blockade had improvement in phenotype. One placebo-treated patient improved spontaneously, while the other had a decline in phenotype. In the 7 subjects who exhibited improvement in phenotype, gene expression was subdivided into functional categories and the number of genes exhibiting an increase or a decrease was determined by standard Affymetrix algorithms that were tailored to the degree of scaling factor required to read each chip.

Gene categories exhibiting decreased expression included growth factors, extracellular matrix proteins, transcription and translation factors, signal transduction proteins, immunologic/hematologic factors, fetal forms of contractile proteins, and some unknown genes. Metabolic genes were the only category of genes the exhibit more increases than decreases in expression. These findings support the conclusion that improvement in IDC phenotype is characterized by a preferential decrease in the expression of genes that contribute to hypertrophy and remodeling. These data indicate that the remodeled, failing human heart is in an activated state of gene expression in order to sustain/advance the remodeling process.

A composite method of data presentation was devised whereby genes were identified that exhibited a change in 4 of the 7 improved subjects or in every subject within a particular type of analysis. Genes were shown to exhibit an increase or decrease in expression by functional gene category. A total of 17 genes exhibited increased expression, while 136 decreased their expression as the phenotype improved. Genes expected to be involved in the remodeling process,such as transcription and translation factors, signal transducers, growth factors and extra-cellular matrix proteins, exhibited the greatest number of decreased expression, disproportional to increased expression. The general method may be a valuable tool for molecular discovery of novel genes/mechanisms involved in the pathophysiology of dilated cardiomyopathy and chronic heart failure.

Endomyocardial biopsies were obtained during a 6-7 month period from eight subjects with baseline idiopathic dilated cardiomyopathy (IDC). Of these subjects, six were treated with the β-blocking agents carvedilol and metoprolol, and two were treated with placebo. All six subjects in the first group showed improved left ventricular ejection fraction (LVEF). Of the two subjects in the placebo group, one showed an improved left ventricular ejection fraction whereas the other a deteriorated LVEF.

Biopsied samples obtained from these subjects were analyzed by quantitative reverse transcript polymerase chain reaction (QRT PCR), and the mRNAs obtained were further analyzed by Affymetrix Gene chip. Subjects with ideal substantial noise ratios were categorized in the low scaling factor group and those with substantial noise, which compromised quantitation, categorized in the high scaling factor group. Gene expression patterns were further compared between the seven subjects showing improved phenotypes and the one subject showing worsening phenotype. Of the four subjects in the low scaling group showing an improved LVEF, one had received placebo, one carvedilol, and one metoprolol, with the fourth subject treated with placebo showing a decrease LVEF. Of the four subjects in the high scaling group three received carvedilol and one received metoprolol.

### Example 2

### Results

Gene expression patterns were compared between the three subjects showing improved LVEF and the one subject showing worsening LVEF in the low scaling group. Of the samples obtained from this group, a number of genes were found to be differentially regulated in diseased endomyocardial tissue versus that of the non-diseased tissue. These results are summarized in Table 1. The results demonstrate the advantage of dynamic expression profiling as a powerful tool for measuring disease-specific phenotypes in gene expression superimposed on identical genetic backgrounds, and in successfully treated subjects.

Serial measurements of myocardial gene expression were performed on 4 subjects with ICM, at baseline and after 6 months of treatment with β-blocking agents (n=2, 1 carvediol and 1 metroprolol) or placebo (n=2). RNA was extracted from endomyocardial biopsy material, and expression profiling was performed via Affymetrix U95a GeneChips. Both β-blocker-treated patients and one placebo-treated patient had improvement in phenotype, while one placebo-treated patient had a decline. In the three subjects who improved, a between-subject anaylsis was performed at baseline, and compared to a within-subject comparison between baseline and end of study.

The results showed greater varation in gene expression between subjects with the same phenotype than within a single individual comparing an advanced disease to marked improvement in disease. These data suggest that serial sampling before and after modulation of phenotype will be a valuable method of molecular discovery, and an improvement on cross-sectional approaches.

A collective analysis of the data includes the following filters: (1) genes that exhibit changes in expression in the majority (i.e., ≧4/7 subjects with improvement in LVEF that averaged (EF Units±SD) 24.1±9.7, (baseline 21.9±9.2; follow-up at 6-7 months 46.0±8.1, paired t p value=0.0006)), or (2) genes that exhibit changed expression in 100% of subjects within an analysis group (in low scaling factor chip pairs 3/3 by Diff Call or Abs Call, in high scaling factor chips 4/4 by Abs Call), and (3) genes that exhibit changes as defined by (1) or (2) and have no change or the opposite directional change in the subject who exhibited a decrease (from 26 to 15) in LVEF. Applying these criteria, one can observe that the number of genes exhibiting decreased expression as the left ventricle phenotype normalizes greatly exceeds the number demonstrating an increase, by 7.7-fold (116 decreases, 15 increases). Viewing the data from the perspective of the baseline data analysis being from severely failing, dilated human left ventricles and the follow-up data having been taken from nonfailing, normalized ventricular phenotypes, it can be appreciated that the failing ventricles are in an “activated state” of gene expression, with nearly 8 times as many genes exhibiting higher expression. This degree of difference in the number of genes exhibiting increased or decreased expression in the nonfailing, normalized left ventricle vs. failing human left ventricle has not been observed in gene chip analyses performed by cross-sectional designs by us or others, where using Affymetrix GeneChip analysis the difference between up-regulated and down-regulated genes in failing vs. nonfailing human ventricles has been respectively 1.1- and 1.7-fold (Tan et al., 2002). This discrepancy highlights the improved precision of measuring gene expression serially with modulation of phenotype.

An examination of the categories of genes exhibiting an increase reveals that the cytoskeletal, extracellular matrix, transcription/translation factor, signal transduction, growth factor, apoptosis/cell cycle, unclassified and unknown function gene categories exhibited decreased expression as the dilated cardiomyopathy (DCM) phenotype normalized. All these gene categories exhibited a marked imbalance towards up-regulation in severe DCM, and the findings are consistent with a molecular profile that would produce pathologic hypertrophy. In addition, within the contractile protein categories 2 fetal isoforms (skeletal isoforms tropomyosin or troponin I) exhibited decreased expression as the phenotype normalized, and the adult (α) isoform of myosin heavy chain (MyHC), which directly leads to improved contractile function in animal models and likely in humans, exhibited increased expression with normalization of phenotype, as well as decreased expression in the left ventricle that deteriorated. The only gene category that exhibited a quantitative increase in expression with normalization of phenotype was the metabolic category, with 4 genes showing increased expression and 2 genes showing decreased expression with normalization of phenotype.

With regard to changes in individual genes within categories, some of those identified are known to be associated with or directly involved with the development of the DCM phenotype. Examples would include α-MyHC, known to be down-regulated in the failing human left ventricle and closely associated with phenotypic improvement (Lowes et al., 2002); MEK5, which when expressed in activated form can cause a dilated cardiomyopathy in transgenic mice (Nicol et al., 2001), and which exhibited decreased expression with phenotypic improvement; and the extracellular matrix (ECM) producing or regulating genes shown to be decreased with phenotypic normalization (n=6), as multiple ECM genes have been shown to have increased expression in DCM (Spinale et al., 2000).

In addition, this method has the ability of detecting novel mechanisms likely to be associated with the DCM phenotype. For example, a Shaker-type, delayed rectifier (Kv1.1) voltage-sensitive potassium channel beta subunit (β1, or KCNA1B; Leicher et al., 1996) was increased with normalization of phenotype, and exhibited decreased expression in the subject with a progression in phenotype. This subunit can markedly alter function of voltage-gated potassium channel α-subunits, and potassium channel delayed rectifier current is markedly dysregulated in the failing heart (Nabauer and Kaab, 1998). When Kv1.1 is genetically disrupted in the heart an arrhythmia phenotype reminiscent of DCM develops, namely prolongation of the action potential and ventricular tachycardia (London et al., 1998). Thus therapeutic strategies to increase function or expression of a down-regulated Kv1.1 β1 subunit would be a logical treatment approach to preventing sudden death in the failing, dilated and hypertrophied human heart. Other examples derive from the specific genes which decreased expression during normalization of phenotype; although in some cases related family members have been shown to be up-regulated in the failing human heart (Spinale et al., 2000), with the exception of Collagenase IV (also known as MMP2) none of the specific genes identified in this analysis have been shown to have increased expression in the failing human heart. Consequently, any or all of them would be candidates for inhibition strategies as therapeutic approaches in decrease pathologic remodeling. Finally, the observation that the metabolic gene category was the only one exhibiting a net increase in expression with phenotypic normalization suggests that therapeutic enhancement of the activites or gene expression of the Na/glucose co-transporter (SGLUT1), long-chain Acyl-CoA Synthase, or a transketolase-like protein would have therapeutic utility in DCM/heart failure.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

| | Accession | | Accession |
|---|---|---|---|
| Increased | # | Decreased | # |
| dioxin inducible | U03688 | apolipoprotein AI | X02162 |
| cytochrome P450 | | | |
| Na/Glucose co- | L29339 | Lysosomal alpha- | U60899 |
| transporter | | mannosidase (LAMAN) | |
| (SGLT1) | | | |
| transketolase-like | X91817 | serine/threonine kinase iso- | AF005046 |
| prtn | | lated from SK-N-MC | |
| | | neuroblastoma cDNA | |
| | | library” | |
| long-chain acyl- | D10040 | | |
| CoA Synthase | | | |
| HepG2 glucose | K03195 | | |
| transporter | | | |
| n = 5 | | n = 3 | |
| a-MyHC | Z20656 | skeletal tropomyosin | X04201 |
| | | skeletal tropomyosin | X04201 |
| | | slow twitch skeletal | J04760 |
| | | troponin I | |
| n = 1 | | n = 2 (skeletal tropomyosin | |
| | | twice) | |
| | | Nebulin | X83957 |
| n = 0 | | plectin (PLEC1) | U53204 |
| | | Lamin B2 (LAMB2) | M94362 |
| | | Keratin 16 (KRT16A), an | AF061812 |
| | | intermediate filament. | |
| | | dystrobrevin-epsilon: mem- | U46746 |
| | | ber of dystrophin assoc | |
| | | complex | |
| | | n = 5 | |
| | | procollagen alpha 2V | Y14690 |
| n = 0 | | fibromodulin | U05291 |
| | | chondrotiin sulphate | X15998 |
| | | proteoglycan versican | |
| | | COMP (germline oligomer- | L32137 |
| | | ic matrix protein (COMP)) | |
| | | ACLP (aortic carboxy- | AF053944 |
| | | peptidase-like protein) | |
| | | Collagenase Type IV | M55593 |
| | | MMP 21/22C | AL031282 |
| | | The thrombospondin and | M92642 |
| | | collagen type I receptor | |
| | | CD36 | |
| | | The thrombospondin and | Z22555 |
| | | collagen type I receptor | |
| | | CD36 | |
| | | Tissue inhibitor of | D11139 |
| | | metalloproteinase (TIMP) | |
| | | n = 9, (1 redundant) | |
| Lsm5 protein. | AJ238097 | Eukaryotic protein synthe- | AF104913 |
| | | sis initiation factor | |
| | | ATBF-1A (zinc finger | L32832 |
| | | homeodomain protein | |
| | | (ATBF1-A) | |
| | | steroid receptor Ner-1 | U07132 |
| | | Transcription factor | AF040253 |
| | | Tat-CT1 | |
| | | repressor protein (HUB1) | D30612 |
| | | glucocorticoid receptor re- | M73077 |
| | | pression factor 1 (GRF-1) | |
| | | region 7 homeobox protein | M97676 |
| | | (HOX7) | |
| | | 3-alkyl adenine DNA | M74905 |
| | | glycosylase (HAAG), a | |
| | | DNA repair enzyme. | |
| | | cAMP response element | L05515 |
| | | binding protein | |
| | | (CRE-BP1) | |
| | | Pur (pur-alpha) | M96684 |
| | | Gamma adaptin. | Y12226 |
| | | Human inteleukin binding | U58198 |
| | | factor 3, a DNA binding | |
| | | protein that binds to | |
| | | NFAT-like motifs | |
| | | NFI/CAAT-binding trans- | X92857 |
| | | cription factor 5 (CTF5). | |
| | | histone H2B | Z80779 |
| n = 1 | | n = 14 | |
| | | calgizzarin | D38583 |
| n = 0 | | n = 1 | |
| STM-7 prtn | X92493 | Protein kinase C zeta | U07358 |
| (Friedreich's | | (zPKC) | |
| Ataxia gene, | | | |
| novel PI 4P 5- | | | |
| kinase) | | | |
| cGMP PDE 5A | D89094 | beta-2 adr receptor | M15169 |
| | | Map kinase kinase | U71087 |
| | | (MEK5b) | |
| | | Regulator of G protein sig- | AF045229 |
| | | naling 10 (RGS10) | |
| | | TBX2 | U28049 |
| | | XAP-4, GDP dissassoci- | X79353 |
| | | ation inhibitor | |
| | | rho GDP dissociation | X69550 |
| | | inhibitor 1 | |
| | | ADP ribosylation-like | U73960 |
| | | protein 4 | |
| | | GABA B R1-a receptor | AJ225028 |
| | | alpha-2 adrenergic receptor | J03853 |
| | | cloned from kidney | |
| | | contactin, a human cell ad- | Z21488 |
| | | hesion molecule. | |
| | | Ndr protein kinase | AF100153 |
| | | tyrosine phosphatase | U27193 |
| | | GTP-binding protein RAB4 | M28211 |
| | | ES/130 | AF006751 |
| | | ES/130 (part of cascade that | AF006751 |
| | | triggers endoth to | |
| | | mesenchme in fetal dvlp, | |
| | | incr in FH) | |
| | | Hepatitis B Virus associ- | U67322 |
| | | ated factor (XAP4). | |
| | | p55PIK, a regulatory sub- | D88532 |
| | | unit of phosphoinositide | |
| | | 3-kinase | |
| | | PDE8A | AF056490 |
| | | Homo sapiens GTP BP | L05425 |
| | | from Breast Ca, autoantigen | |
| n = 2 | | n = 19 (ES130 twice) | |
| Heat Shock prtn | U15590 | Chloride channel CLC-7 | Z67743 |
| (HSPB3) | | | |
| voltage gated | X83127 | 116 kDa vacuolar proton | U45285 |
| potassium chan- | | pump subunit (OC 116kD) | |
| nel, beta subunit | | | |
| (hKvbeta1) | | | |
| | | Heat Shock protein HSPA2 | L26336 |
| | | gene | |
| | | heat shock protein A2 | L26336 |
| | | carbonic anhydrase III | M29458 |
| | | TTAGGG repeat binding | AF003001 |
| | | factor 1 (hTRF1-AS). | |
| n = 2 | | n = 5 (HSPA2 twice) | |
| EEN-B2-L3, | AF036271 | follistatin-related protein | D89937 |
| fused with MLL | | (FRP). (possibly redundant) | |
| gene in human | | | |
| leukemia. | | | |
| | | BRCA2 region on Chr 13, | U50535 |
| | | includes the breast cancer | |
| | | susceptibility locus BRCA2 | |
| | | placental bikunin, a novel | U78095 |
| | | serine protease inhibitor | |
| | | human homologue of yeast | U13695 |
| | | mutL (hPMS1). | |
| | | Lissencephaly (LIS) gene | L25107 |
| | | Pigment epithelium derived | U29953 |
| | | factor (antiangiogenesis | |
| | | factor) | |
| | | retinol binding protein | M11433 |
| | | A novel cDNA clone | M80244 |
| | | termed E16 | |
| | | Human melanoma Ag | U19796 |
| | | Ski-related oncogene | X15218 |
| | | Lissencephaly (LIS) gene. | L25107 |
| | | A novel cDNA clone | M80244 |
| | | termed E16 | |
| | | spermatogenesis associated | AL031685 |
| | | protein 2 | |
| | | SH3-containing adaptor | AF037261 |
| | | molecule-1 | |
| | | Precerebellin. | M58583 |
| n = 1 | | n = 14 (LIS gene twice) | |
| a-globin regula- | X90857 | S-protein | X03168 |
| tory element | | | |
| Clq complement | U94333 | S-protein | X03168 |
| n = 2 | | n = 1 (S-protein twice) | |
| nucleolysm TIA-1 | M96954 | Apoptosis repressor ARC | AF043244 |
| gene | | | |
| | | protein kinase Pitslre alpha | HG4120- |
| | | | HT4392 |
| | | WSL-LR, WSL-S1, WSL- | Y09392 |
| | | S2 proteins, death-domain- | |
| | | containing receptors | |
| | | CDK inhibitor p19INK4d | U40343 |
| | | RIP protein kinase. | U50062 |
| | | DAP-1 | X76105 |
| | | PLSTIRE for serine/ | X66365 |
| | | threonine PK. | |
| | | PCTAIRE-2 for serine/ | X66360 |
| | | threonine protein kinase. | |
| n = 1 | | n = 8 | |
| L(3)mbt protein | U89358 | c-myc binding protein | D50692 |
| (A human homo- | | | |
| log of Drosophila | | | |
| lethal(3)malignant | | | |
| brain tumor) | | | |
| | | RYK, related to receptor | S59184 |
| | | tyrosine kinase (human | |
| | | hepatoma). | |
| | | insulin like growth factor II | X57025 |
| | | (redundant) | |
| | | growth arrest specific | L13720 |
| | | protein (gas) | |
| | | OSF-2os (osteoblast- | D13666 |
| | | specific factor) | |
| | | radiation-inducible imm | S81914 |
| | | early gene | |
| | | hematopoetic neural mem- | U87947 |
| | | brane prtn (HNMP-1) | |
| | | TGFb-3 | X14885 |
| | | GDF1 (human growth dif- | M62302 |
| | | ferentiation factor) | |
| n = 1 | | n = 9 | |
| KIAA1048 | AB028971 | KIAA0459 prtn | AB007862 |
| | | KIAA0459 prtn | AB007862 |
| | | KIAA0402 | AB007862 |
| | | KIAA0459 prtn | AB007862 |
| | | KIAA0402 | AB007862 |
| | | KIAA0402 | AB007862 |
| | | KIAA0730 | AB018273 |
| | | unkn clone | AF044968 |
| | | KIAA0965 | AB023182 |
| | | unkn clone | AL049980 |
| | | KIAA1108 | AB029031 |
| | | 3′ end clone | AI191768 |
| | | unkn full length clone | AL109702 |
| | | 3′ clone | AI813532 |
| | | 3′ clone | AI813532 |
| | | DFKZp564L0822 | AL049949 |
| | | IMAGE-2314914 | AI670100 |
| | | 5′ end clone | AA099265 |
| | | 3′ end clone | AI290660 |
| | | KIAA0739 | AB018282 |
| | | 3′ end clone | N30625 |
| | | unkn clone | AL080134 |
| | | KIAA1003 | AB023220 |
| | | KIAA1003 | AB023220 |
| | | KIAA0426 | AB007886 |
| | | 5′ end clone, unkn | N25117 |
| | | KIAA0015 | D13640 |
| | | KIAA0206 | D86961 |
| | | KIAA025 gene | D86969 |
| | | KIAA1066 | AB028989 |
| | | Clone from Chr 1 q22-24.2, | AL049798 |
| | | incl dermatopontin gene | |
| | | and ESTs | |
| | | KIAA1062 | AB028985 |
| | | KIAA0306 | AB002304 |
| | | chromosome 3p21.1 | L13434 |
| | | KIAA0128 | D50918 |
| | | 3′ end clone | AI991631 |
| | | Unkn clone | U69559 |
| | | KIAA1028 | AB028951 |
| | | IT12, unkn protein | AF040965 |
| | | unkn protein B | U47926 |
| | | 3′ end clone | AI660225 |
| | | KIAA0518 | D63878 |
| | | KIAA0830 | AB020637 |
| | | 3′ end clone, unkn | AI345337 |
| | | KIAA1037 | AB028960 |
| | | unkn clone | AF052497 |
| | | 5′ end clone | AA037278 |
| | | unkn cDNA | AL050022 |
| | | IMAGE-2372101 | AI743134 |
| | | Unkn cDNA | W28742 |
| | | KIAA0870 protein, unkn | AB020677 |
| | | KIAA0459 prtn | AB007928 |
| | | KIAA0402 prtn | AB0077862 |
| n = 1 | | n = 46, 7 redundants | |
| Grand Total, | | Grand Total, n = 136 | |
| n = 17 | | | |

### REFERENCES

The following references, to the extent that they provide exemplary procedural or other details supplementary to those set forth herein, are specifically incorporated herein by reference.

U.S. Pat. No. 5,786,461

U.S. Pat. No. 5,766,855

U.S. Pat. No. 5,719,262

U.S. Pat. No. 5,714,331

U.S. Pat. No. 5,736,336

U.S. Pat. No. 5,891,625

U.S. Pat. No. 5,539,082

U.S. Pat. No. 4,683,195

U.S. Pat. No. 4,683,202

U.S. Pat. No. 4,800,159

U.S. Pat. No. 4,883,750

U.S. Pat. No. 5,168,053

U.S. Pat. No. 5,354,855

U.S. Pat. No. 5,578,832

U.S. Pat. No. 5,604,251

U.S. Pat. No. 5,624,824

U.S. Pat. No. 5,625,047

U.S. Pat. No. 5,837,832

U.S. Pat. No. 5,837,860

U.S. Pat. No. 5,861,242

U.S. Pat. No. 5,908,845

U.S. Pat. No. 5,786,461

U.S. Pat. No. 5,891,625

U.S. Pat. No. 5,773,571

U.S. Pat. No. 5,766,855

U.S. Pat. No. 5,736,336

U.S. Pat. No. 5,719,262

U.S. Pat. No. 5,714,331

U.S. Pat. No. 5,539,082

Baichwal and Sugden, In: Gene Transfer, New York, Plenum Press, 117-148, 1986.

Barnes et al., J. Biol. Chem, 272(17):11510-11517, 1997.

Berzal-Herranz et al., Genes Dev, 6(1):129-134, 1992.

Bhavsar et al., Genomics, 35(1):11-23, 1996.

Bristow, Cardiology, 92:3-6, 1999

British Application No. GB 2,202,328

Cech et al., Cell, 27(3 Pt 2):487-96, 1981.

Chang et al., Mol. Cell. Biol., 9:2153, 1991.

Chen and Okayama, Mol. Cell. Biol., 7(8):2745-2752, 1987.

Chowrira et al., Biochemistry, 32(4):1088-1095, 1993.

Coffin, In: Virology, Fields et al., eds., Raven Press, New York, pp. 1437-1500, 1990.

Coupar et al., Gene, 68:1-10, 1988.

Dubensky et al., Proc. Nat'l Acad. Sci. USA, 81:7529-7533, 1984.

Eichhorn and Circulation, 94:2285-2296, 1996

European Application No. 0273085

European Application No. 320,308

European Application No. 329,822

Fechheimer et al., Proc. Nat'l Acad. Sci. USA, 84:8463-8467, 1987.

Ferkol et al., FASEB J., 7:1081-1091, 1993.

Flanagan et al., Nature, 352:803-807, 1991.

Fodor et al., Nature, 364:555-556, 1993.

Force et al., Circ. Res., 78:947-953, 1996.

Forster and Symons, Cell, 49(2):211-220, 1987.

Fraley et al., Proc. Nat'l Acad. Sci. USA, 76:3348-3352, 1979.

Franz et al., Cardioscience, 5(4):235-43, 1994.

Friedmann, “Progress toward human gene therapy”, Science, 244:1275-1281, 1989.

Frohman, In: PCR Protocols: A Guide To Methods And Applications, Academic Press, N.Y., 1990.

Ghosh and Bachhawat, In: Liver Diseases, Targeted Diagnosis and Therapy Using Specific Receptors and Ligands. Marcel Dekker, New York, pp. 87-104, 1991.

Ghosh-Choudhury et al., EMBO J., 6:1733-1739, 1987.

Gomez-Foix et al., J. Biol. Chem., 267:25129-25134, 1992.

Goodman and Gilman's The Pharmacological Basis of Therapeutics

Gopal, Mol. Cell Biol., 5:1188-1190, 1985.

Gopal-Srivastava et al., Mol Cell Biol 15(12):7081-90, 1995.

Graham and Prevec, Biotechnology, 20:363-390, 1992.

Graham and Prevec, In: Methods in Molecular Biology: Gene Transfer and Expression Protocol, Humana Press, Clifton, N.J., 7:109-128, 1991.

Graham and van der Eb, Virology, 52:456-467, 1973.

Graham, et. al., J. Gen. Virl., 36(1):59-74, 1977.

Grunhaus et al., Seminar in Virology, 200(2):535-546, 1992.

Hacia, et al., Nature Genet., 14:441-449, 1996.

Harland and Weintraub, J. Cell Biol., 101 :1094-1099, 1985.

Haseloff and Gerlach, Nature, 334(6183):585-91, 1988.

Hermonat and Muzycska, Proc. Nat'l Acad. Sci. USA, 81:6466-6470, 1984.

Hersdorffer et al., DNA Cell Biol., 9:713-723, 1990.

Herz and Gerard, Proc. Nat'l Acad. Sci. USA, 90:2812-2816, 1993.

Holmstrom et al., Anal. Biochem. 209:278-283, 1993.

Horwich et al., Virol., 64:642-650, 1990.

Jones and Shenk, Cell, 13:181-188, 1978.

Joyce, “RNA evolution and the origins of life,” Nature, 338:217-244, 1989.

Kaneda et al., Science, 243:375-378, 1989.

Karlsson et al., EMBO J., 5:2377-2385, 1986.

Kato et al., J. Biol. Chem., 266:3361-3364, 1991.

Kelly et al., J Cell Biol, 129(2):383-96, 1995.

Kim and Chech, Proc. Natl. Acad. Sci. USA, 84(24):8788-8792, 1987.

Kirura et al., Anesthesiol. Cli., 33:21-37, 1995.

Klein et al., Nature, 327:70-73, 1987.

Kwoh et al., Proc. Nat. Acad. Sci. USA, 86: 1173, 1989.

LaPointe et al., Hypertension 27(3 Pt 2):715-22, 1996.

Le Gal La Salle et al., Science, 259:988-990, 1993.

Leicher et al. “Structural and functional characterization of human potassium channel subunit β1 (KCNA1B),” Neuropharmacology 35:787-795, 1996.

Levrero et al., Gene, 101:195-202, 1991.

Loh et al., J. Biol. Chem., 271:10884-10891, 1996.

Loh et al., Mol. Cell. Biol., 16:3945-3954, 1996.

London et al., “Long QT and ventricular arrhythmias in transgenic mice expressing the N terminus and first transmembrane segment of a voltage-gated potassium channel,” Proc. Nat'l Acad. Sci. USA 95:2926-2931, 1998.

Lowes et al., “Myocardial gene expression in dilated cardiomyopathy treated with beta-blocking agents,” New Engl. J. Med. 346:1357-1365, 2002.

Macejak and Sarnow, Nature, 353:90-94, 1991.

Mann et al., Cell, 33:153-159, 1983.

Markowitz et al., J. Virol., 62:1120-1124, 1988.

Michel and Westhof, J. Mol. Biol., 216:585-610, 1990.

Moss et al., J Biol Chem, 271(49):31688-94, 1996.

Mulligan, Science, 260:926-932, 1993.

Nabauer and Kaab, “Potassium channel down-regulation in the heart,” Cardiovasc Res. 37:324-334, 1998.

Newton, et al. Nucl. Acids Res. 21:1155-1162, 1993.

Nicol et al., “Activated MEK5 induces serial assembly of sarcomeres and eccentric cardiac hypertrophy,” EMBO J. 20:2757-2767, 2001.

Nicolas and Rubinstein, In: Vectors: A survey of molecular cloning vectors and their uses, Stoneham: Butterworth, pp. 494-513, 1988.

Nicolau and Sene, Biochim. Biophys. Acta, 721:185-190,1982.

Nicolau et al., Methods Enzymol., 149:157-176, 1987.

Ohara et al., Proc. Nat'l Acad. Sci. USA, 86: 5673-5677, 1989.

Paskind et al., Virology, 67:242-248, 1975.

PCT Application No. PCT/US87/00880

PCT Application No. PCT/US89/01025

PCT Application No. WO 88/10315

PCT Application No. WO 89/06700

PCT Application No. WO 90/07641

PCT Application No. WO 98/33791

PCT Application No. WO 92/20702.

PCT Application No. EP/01219.

Pease et al., Proc. Natl. Acad. Sci. USA, 91:5022-5026, 1994.

Pelletier and Sonenberg, Nature, 334:320-325, 1988.

Perales et al., Proc. Nat'l Acad. Sci., 91:4086-4090, 1994.

Perriman et al., Gene, 113(2):157-163, 1992.

Physicians Desk Reference

Potter et al., Proc. Nat'l Acad. Sci. USA, 81:7161-7165, 1984.

Racher et al., Biotechnology Techniques, 9:169-174, 1995.

Ragot et al., Nature, 361:647-650, 1993.

Rao et al., Annu. Rev. Immunol., 15:707-747, 1997.

Rasmussen, et al., Anal. Biochem, 198:138-142, 1991.

Reinhold-Hurek and Shub, Nature, 357:173-176, 1992.

Remington's Pharmaceutical Sciences and The Merck Index, 11th Edition

Renan, Radiother. Oncol., 19:197-218, 1990.

Ridgeway, In: Vectors: A survey of molecular cloning vectors and their uses, Stoneham: Butterworth, pp. 467-492, 1988.

Rippe et al., Mol. Cell. Biol., 10:689-695, 1990.

Ritchie, M. E., J. Biol. Chem. 271(41):25485-25491, 1996.

Rosenfeld et al., Science, 252:431-434, 1991.

Rosenfeld et al., Cell, 68:143-155, 1992.

Roux et al., Proc. Nat'l Acad. Sci. USA, 86:9079-9083, 1989.

Running et al., BioTechniques 8:276-277, 1990.

Sadoshima et al., Circ. Res., 73:424-438, 1993.

Sambrook et. al., In: Molecular Cloning: A Laboratory Manual, 2d Ed., Cold Spring Harbor Laboratory Press, Cold Spring Harbor, N.Y., 1989.

Shoemaker et al., Nature Genetics 14:450-456, 1996.

Spinale et al., “A matrix metallotroteinase induction/activation system exists in the human left ventricular myocardium and is upregulated in heart failure,” Circulation 102:1944-1949, 2000.

Stratford-Perricaudet et al., Hum. Gene. Ther., 1:241-256, 1990.

Stratford-Perricaudet et al., Hum. Gene. Ther., 1:241-256, 1990.

Stratford-Perricaudet and Perricaudet, In: Human Gene Transfer, John Libbey Eurotext, France, pp. 51-61, 1991.

Symons, “Small catalytic RNAs.” Annu. Rev. Biochem., 61:641-671, 1992.

Tan et al., “The gene expression fingerprint of human heart failure,” Proc. Nat'l Acad. Sci. USA 99:11387-11392, 2002.

Temin,In: Gene Transfer, New York: Plenum Press, pp. 149-188, 1986.

Thompson et al., “Ribozymes in gene therapy.” Nature Medicine, 1:277-278, 1995.

Top et al., J. Infect. Dis., 124:155-160, 1971.

Tur-Kaspa et al., Mol. Cell. Biol., 6:716-718, 1986.

Varmus et al., Cell, 25:23-36, 1981.

Wagner et al., Proc. Nat'l Acad. Sci. USA 87(9):3410-3414, 1990.

Walker et al., Proc. Nat'l Acad. Sci. USA, 89:392-396 1992.

Wong et al., Gene, 10: 87-94, 1980.

Wu and Wu, J. Biol. Chem., 262:4429-4432, 1987.

Wu and Wu, Biochemistry, 27:887-892, 1988.

Wu and Wu, Adv. Drug Delivery Rev., 12:159-167, 1993.

Yamauchi-Takihara, et. al., Proc. Nat'l Acad. Sci. USA 86(10):3504-3508, 1989.

Yamazaki et al., J. Biol. Chem., 271:3221-3228, 1996.

Yang et al., Proc Nat'l Acad Sci. USA, 87:9568-9572, 1990.

Yuan and Altman, Science, 263:1269-1273, 1994.

Yuan et al., Proc. Natl. Acad. Sci. USA, 89:8006-8010, 1992.

Zarian-Herzberg et al., Am. J. Cardiol., 93:31H-37H, 1999.

Zelenin et al., FEBS Lett., 280:94-96, 1991.

Ziobe and Kramer, J. Biol. Chem, 271(37):22915-22, 1996.

Zou et al., J. Biol. Chem., 271:33592-33597, 1996.

What is claimed is:

1. A method of identifying gene involvement in the development, progression and/or maintenance of a disease state comprising:
(a) obtaining a nucleic acid-containing sample from a first subject suffering from the disease state;
(b) obtaining a histologically similar nucleic acid-containing samples from (i) a subject suffering from the disease state, wherein the subject has received a therapy that ameliorates the phenotype of the disease state, and (ii) a subject suffering from the disease state, wherein the subject has received a therapy that does not ameliorate the phenotype of the disease state;
(c) obtaining gene expression profiles from the samples in steps (a) and (b); and
(d) comparing the gene expression profiles in steps (a) and (b);
wherein a gene whose expression increases or decreases in the sample of step (b)(i), as compared to the samples of steps (a) and (b)(ii), is identified as being involved in the development, progression and/or maintenance of the disease state.

2. The method of claim 1, further comprising:
(e) obtaining a second nucleic acid-containing sample from the subject of step (b)(i) from at least one later time point;
(f) obtaining a gene expression profile from the sample in step (e); and
(g) comparing the gene expression profile in step (f) to the gene expression profile in step(b)(i).

3. The method of claim 1, wherein the disease state is heart failure, cancer, obesity, a neurodegenerative disease, kidney failure, and liver failure.

4. The method of claim 1, wherein the nucleic acid-containing sample is a tissue sample.

5. The method of claim 1, wherein obtaining expression profiles comprises PCR.

6. The method of claim 5, wherein PCR comprises RT-PCR.

7. The method of claim 1, wherein obtaining expression profiles comprises use of a gene array disposed on a chip.

8. The method of claim 3, wherein the disease state is heart failure.

9. The method of claim 1, further comprising comparing the gene expression profile from the samples in steps (a), (b)(i) and/or (b)(ii) with the gene expression profile of a subject suffering from the disease state receiving placebo rather than therapy.

10. The method of claim 1, further comprising comparing the gene expression profile from the samples in steps (a), (b)(i) and/or (b)(ii) with the gene expression profile of a histologically similar sample from a healthy individual.

11. The method of claim 1, further comprising repeating steps (a)-(d) with at least a second subject suffering from the disease state, and comparing the results obtained with the first subject.

12. A method of identifying gene involvement in the development, progression and/or maintenance of a disease state of an individual comprising:
(a) obtaining a nucleic acid-containing sample from a subject suffering from the disease state;
(b) obtaining a histologically similar nucleic acid-containing sample from the subject of step (a) from at least one later time point, prior to which the subject has received a therapy that ameliorates the phenotype of the disease state;
(c) obtaining gene expression profiles from the samples in steps (a) and (b); and
(d) comparing the gene expression profile from the samples in steps (a) and (b),
wherein a gene whose expression increases or decreases in the sample of step (b), as compared to the samples of step (a), is identified as being involved in the development, progression and/or maintenance of the disease state.

13. The method of claim 12, further comprising repeating step (b) at a second later time point.

14. The method of claim 12, further comprising
(e) obtaining a histologically similar nucleic acid-containing sample from a subject suffering from the disease state, wherein the subject has received a therapy that does not ameliorate the phenotype of the disease state;
(f) obtaining a gene expression profile from the sample in step (e); and
(g) comparing the gene expression profile in step (f) to the gene expression profile in step(b).

15. A method for assessing the efficacy of a cardiac disease therapy comprising:
(a) obtaining a first cardiac tissue sample from a first subject suffering from a cardiac disease;
(b) treating the first subject with a candidate therapy;
(c) obtaining a second cardiac tissue sample from the first subject following treatment; and
(d) comparing the expression of one or more indicator genes in the first and second samples, the one or more indicator genes as listed in Table 1,
wherein a change in the expression of one or more indicator genes indicates that the candidate therapy is effective at treating cardiac disease in the first subject.

16. The method of claim 15, wherein the indicator gene is upregulated.

17. The method of claim 15, wherein the indicator gene is downregulated.

18. The method of claim 15, wherein obtaining expression profiles comprises PCR.

19. The method of claim 18, wherein PCR comprises RT-PCR.

20. The method of claim 15, wherein obtaining expression profiles comprises use of a gene array disposed on a chip.

21. The method of claim 15, further comprising comparing the gene expression profile from the samples in steps (a) and/or (b) with the gene expression profile of a cardiac tissue sample from a healthy individual.

22. The method of claim 15, further comprising comparing the gene expression profile from the samples in steps (a) and/or (b) with the gene expression profile of a cardiac tissue sample from a second subject suffering from cardiac disease receiving a placebo rather than therapy.

23. The method of claim 15, further comprising repeating steps (a)-(d) with at least a second subject suffering from cardiac disease, and comparing the results obtained with the first subject.

24. The method of claim 23, further comprising repeating steps (a)-(d) on the first subject after altering the dose or dosing regimen of the candidate therapy.

25. A method of screening a candidate substance for their ability to modulate the activity of one or more cardiac disease gene products in cardiac cells comprising:
(a) providing a myocyte;
(b) contacting the myocyte with the candidate substance; and
(c) measuring the activity of one or more gene products selected from the group consisting of gene products listed in Table 1,
wherein a change in the activity of one or more gene products selected from the group consisting of Table 1, as compared to the activity in a myocyte not contacted with the candidate substance, indicates that the candidate substance is a modulator of the activity of one or more cardiac disease gene products.

26. The method of claim 25, wherein measuring the activity comprises measuring mRNA levels.

27. The method of claim 26, wherein measuring mRNA levels comprises RT-PCR.

28. The method of claim 25, wherein measuring the activity comprises measuring protein levels.

29. The method of claim 25, wherein measuring the activity comprises measuring enzyme activity.

30. The method of claim 25, wherein the myocyte is a cardiomyocyte.

31. The method of claim 25, wherein the myocyte is contacted in culture.

32. The method of claim 25, wherein the myocyte is contacted in a non-human animal.

33. The method of claim 25, wherein the myocyte has been transformed with an expression construct comprising a screenable marker gene under the control of a promoter derived from a gene selected from Table 1.

34. The method of claim 25, wherein the myocyte exhibits cardiac disease-like gene expression patterns.

35. A method for treating cardiac disease comprising administering to a subject in need thereof a substance that inhibits the activity of one or more of the down-regulated gene products listed in Table 1.

36. The method of claim 35, wherein the substance is a protein.

37. The method of claim 35, wherein the substance is a nucleic acid expression construct.

38. The method of claim 37, wherein the nucleic acid expression construct encodes an antisense construct or ribozyme.

39. The method of claim 35, wherein the substance is a small molecule or organo-pharmaceutical.

40. A method for treating cardiac disease comprising administering to a subject in need thereof a substance that increases the activity of one or more of the upregulated gene products in Table 1.

41. The method of claim 40, wherein the substance is a protein.

42. The method of claim 40, wherein the substance is a nucleic acid expression construct.

43. The method of claim 42, wherein the nucleic acid expression construct encodes one or more of upregulated gene products in Table 1.

44. The method of claim 40, wherein the substance is a small molecule or organo-pharmaceutical.
